(19) United States
(12) Patent Application Publication
(10) Pub. No.: US 2025/0242086 A1
(43) Pub. Date: Jul. 31, 2025

(54) THERAPEUTIC BIOMATERIAL THAT ATTENUATES THE FOREIGN BODY RESPONSE

(71) Applicant: The Regents of the University of Colorado, a body corporate, Denver, CO (US)

(72) Inventors: Stephanie Bryant, Boulder, CO (US); Joshita Suresh, Birmingham, AL (US); William Janssen, Greenwood Village, CO (US); Alexandra McCubbrey, Denver, CO (US)

(73) Assignee: The Regents of the University of Colorado, a body corporate, Denver, CO (US)

(21) Appl. No.: 18/862,541

(22) PCT Filed: May 8, 2023
(86) PCT No.: PCT/US2023/021354

§ 371 Date: Nov. 3, 2024

Related U.S. Application Data
(60) Provisional application No. 63/364,346, filed on May 8, 2022.

(51) Int. Cl. A61L 27/54; A61L 27/34; A61L 27/52

(52) U.S. Cl. CPC A61L 27/54; A61L 27/34; A61L 27/52; A61L 2300/432; A61L 2300/606; A61L 2300/62

(57) ABSTRACT

Systems and methods to eliminate or reduce the foreign body response (FBR) that occurs when a medical device is implanted into a patient. The FBR causes a chronic inflammatory response that leads to the encapsulation of a medical device by a fibrous capsule. Macrophages have been discovered to become persistent as a result of the implanted biomaterial which occurs by an up-regulation in cFLIP. This persistence of macrophages appears to be the primary driver of the FBR. Re-sensitizing macrophages to apoptosis using a small molecule inhibitor (e.g., YM155) of cFLIP will abrogate the formation of the fibrous capsule in the FBR.

### CROSS-REFERENCE TO RELATED APPLICATIONS

This application claims the benefit of U.S. Provisional Application No. 63/364,346 filed May 8, 2022.

### STATEMENT OF GOVERNMENT INTEREST

This invention was made with government support under grant number R21 EB029261 awarded by the National Institutes of Health. The government has certain rights in the invention.

### FIELD OF INVENTION

This invention relates to implantable medical devices. More specifically, this invention relates to methods and compositions or coatings for preventing foreign body response to implantable medical devices.

### BACKGROUND OF THE INVENTION

When a medical device is implanted into the body, it elicits a foreign body response (FBR). The FBR is driven by the innate immune system, namely macrophages, and is characterized by the encapsulation of the device with a dense fibrous capsule. This fibrous capsule is avascular and aneural and thus limits communication and integration with the surrounding tissue. Success of current FDA-approved implantable medical devices relies on the body's ability to tolerate the devices and on the ability of the device to function despite a fibrous capsule. Despite successes of implantable medical devices, the FBR is responsible for aseptic device failure. One example is orthopedic implant loosening that is attributed to poor integration caused by the FBR. Moreover, the FBR has been a significant hurdle to many medical devices that require sensing (e.g., glucose sensors for diabetic patients). To date, there is no therapy or therapeutic biomaterial that is capable of preventing the FBR.

### SUMMARY OF THE INVENTION

The global market for implantable medical devices was $92 million in 2020 and is estimated to grow to $180 million by 2030. In 2018, there were nearly 11 million devices implanted into humans in the U.S. alone. As defined by the FDA, an implantable medical device is a device that is placed into the human body for a period of 30 days or more. This invention has identified a therapeutic target (i.e., cFLIP in macrophages), which could be targeted by a small molecule that is incorporated into a coating of an implant. The approach taught herein targets a novel pathway not previously known to be up-regulated in macrophages in the FBR.

The present invention leverages the discovery that the molecule cFLIP in macrophages is responsible for their persistence at the surface of an implant. cFLIP inhibits the apoptosis pathway and prevents a cell from undergoing programmed cell death. cFLIP was discovered in cancer cells. Surprisingly, it was found that inhibiting cFLIP in macrophages attenuated the formation of a fibrous capsule. We identified this mechanism using a novel transgenic mouse model where cFLIP is conditionally knocked down in macrophages, but not in other cell types. This discovery indicates that a primary driver of the FBR is due to the macrophage becoming resistant to apoptosis and thus disrupting its normal wound healing response. This discovery identifies a novel pathway not previously known, which can be used to target macrophages in the FBR. Importantly, this pathway does not impair the inflammatory response of macrophages, which is known to be critical in wound healing and is also important to minimize any potential device-related infection. We envision that a biomaterial or a biomaterial coating that contains a releasable small molecule that inhibits cFLIP will sensitize macrophages to death signals in the environment, allowing them to undergo apoptosis as in normal wound healing. As a result, the fibrous capsule around a medical device would be reduced. The present invention leverages this newly discovered pathway in the FBR and targeting the pathway as a means to prevent the FBR to implantable medical devices.

This invention has the potential to make a significant impact on the medical device field. By preventing encapsulation of implanted medical devices, this new therapeutic approach would open the door for both extending the life of current medical devices as well as opening the door for new medical devices that require long-term communication and integration with surrounding tissue.

This invention will address broadly the medical device field. Implantable medical devices are currently limited to devices that can either function with a fibrous capsule or that are short-term implants (i.e., only need to function for 1-3 weeks before the device becomes encapsulated). This invention could potentially open the door for many more medical devices to be implanted because they will be able to function longer if there is no fibrous capsule, or a limited fibrous capsule, is present.

The present invention provides systems and compositions to address the formidable problem of the foreign body response (FBR) that occurs when a medical device is implanted into a patient. This FBR causes a chronic inflammatory response that leads to the encapsulation of the device by a fibrous capsule. We recently discovered that macrophages become persistent as a result of the implanted biomaterial which occurs by an up-regulation in cFLIP. This persistence of macrophages appears to be the primary driver of the FBR. Re-sensitizing macrophages to apoptosis using a small molecule inhibitor of cFLIP will abrogate the formation of the fibrous capsule in the FBR.

To provide systems to screen for compounds that attenuate the FBR, (1) development of an in vitro macrophage cell line that over expresses cFLIP can be generated, (2) known small molecule inhibitors of cFLIP can be screened using this macrophage cell line, and (3) a mouse model as taught herein can be used to demonstrate a therapeutic biomaterial with controlled release of the inhibitor that attenuates the FBR in vivo using this mouse model. A first approach is to investigate whether continuous exposure of a low-level inflammatory stimulant over the course of a week or longer causes an up-regulation in cFLIP protein in macrophages and in turn makes the cells resistant to a death signal. This experimental approach will serve as the in vitro screening platform for small molecule inhibitors. Another approach is to use genetic engineering to alter the macrophage cell line to over-express cFLIP.

cFLIP inhibitors for cancer cells can also be used. cFLIP has been identified in cancer cells, which leads to their ability to escape cell death. Tethering the small molecule inhibitor to a biomaterial via a degradable linker can be used demonstrate the ability of a therapeutic biomaterial to attenuate the FBR using the mouse model taught herein. This can be compared to a transgenic mouse model that targets deletion of cFLIP specifically in macrophages.

It is contemplated that the FBR can be limited by delivering a cFLIP inhibitor, such as YM155, to the site of implantation of a device. The cFLIP inhibitor can be delivered in a complex with an additional biomaterial to facilitate retention and subsequent release at the desired site (e.g., the site of implantation of a device). By way of example, two coating strategies can be employed that leverage the demonstration herein that a cFLIP inhibitor can be used to limit the FBR.

The first coating strategy is direct surface modification with the inhibitor. A variety of surfaces are amenable to exposure to oxygen plasma to create radicals, which can then be reacted with a molecule like silane. Using a difunctional molecule, such as Acrylate-PEG_{3,4k}-Silane, this method allows the silane to react with the surface of the biomaterial to form acrylates on the surface. Acrylates can be reacted with other molecules such as thiols through a Michael-type addition click reaction. By conjugating the inhibitor to a linker (such as poly(ethylene glycol) and then to a peptide containing a cysteine (which has a free thiol) or other thiol containing molecule, the inhibitor can be immobilized onto the surface. The linker and/or peptide can be designed to degrade releasing the inhibitor. Degradation could be through enzymes that cleave a specific peptide sequence or by hydrolytically degradable bond.

A second coating strategy is encapsulation. The same method described above to functionalize a surface with acrylates can be used Acrylates can undergo chain or step-growth polymerization when reacted with a multifunctional acrylate or multifunctional PEG monomers to produce a polymeric coating. In the presence of the inhibitor this can lead to the encapsulation of the inhibitor in the polymer coating. The inhibitor is then released by diffusion through the coating. Alternatively, the inhibitor could be conjugated to the polymer coating through a degradable linker. The inhibitor is only released when the linker is degraded. Degradation could be through enzymes that cleave a specific peptide sequence or by hydrolytically degradable bond.

Similarly, a cFLIP inhibitor could be combined with a hydrogel. In this manner the hydrogel is the biomaterial. Within the context of a hydrogel, the coating can be through encapsulation via a degradable tether. Hydrogels are formed by polymerization of multifunctional monomers. Examples include multi-functional acrylates or methacrylates by chain polymerization and multifunctional norbornene, maleimide, or acrylates that react with multifunctional thiols through a step-growth mechanism. The inhibitor can be conjugated to a reactive group (e.g., acrylate, methacrylate, norbornene, thiol) via a linker that can be designed to degrade, such as a peptide or hydrolytically degradable bond. The inhibitor is then released when the linker degrades by an enzyme or by water, respectively.

In certain aspects the present invention provides a method of inhibiting or reducing the FBR responsive to the implantation of a medical device by delivering to the site of implantation one or more small molecule inhibitors of cFLIP. In an advantageous embodiment the small molecule inhibitor is YM155.

In a second aspect the present invention provides a coating for an implantable medical device. The coating can include the small molecule inhibitor YM155 and an encapsulating agent or hydrogel. The encapsulating agent or hydrogel is advantageously one that is capable of immobilization on the surface of an implantable medical device. The YM155 can be conjugated to the encapsulating agent to tether it to the agent, with its sustained release over time as the linkage is broken. Advantageously, the encapsulating agent is a biodegradable polymer. In a particularly advantageous embodiment the encapsulating agent is an acrylate. The coating according to the second aspect can be used in methods of preparing a medical device for implantation, including the step of coating the medical device with the coating according to the second aspect.

In a third aspect the present invention provides an additional coating for an implantable medical device. The coating can include a small molecule inhibitor of survivin. In an advantageous embodiment small molecule inhibitor of surviving is YM155, FL118, SF002-96-1, Terameprocol, WM-127, GDP366, Abbot 8, LLP3, LLP9, S12, Indinavir, Nelfinavir, LQZ-7, LQZ-7F, LQZ-7I, Shepherdin, AICAR, Deazaflavin analog compound 1, UC-112, MX-106, Compound 12b, Compound 10f, Compound 10h, Compound 10k, Compound 10n, PZ-6-QN (and combinations thereof) and an encapsulating agent or hydrogel. The encapsulating agent or hydrogel is advantageously one that is capable of immobilization on the surface of an implantable medical device. The small molecule inhibitor of survivin can be conjugated to the encapsulating agent to tether it to the agent, with its sustained release over time as the linkage is broken. Advantageously, the encapsulating agent is a biodegradable polymer. In a particularly advantageous embodiment the encapsulating agent is an acrylate. The coating according to the third aspect can be used in methods of preparing a medical device for implantation, including the step of coating the medical device with the coating according to the third aspect.

In a fourth aspect the present invention provides an additional coating for an implantable medical device. The coating can include a small molecule inhibitor of cFLIP and an encapsulating agent or hydrogel. The encapsulating agent can be one that is capable of immobilization on the surface of an implantable medical device. The small molecule inhibitor of cFLIP can be YM155 or an analog thereof. The coating according to the fourth aspect can be used in methods of preparing a medical device for implantation, including the step of coating the medical device with the coating according to the fourth aspect.

In a fifth aspect the present invention provides a method of inhibiting or reducing the FBR responsive to the implantation of a medical device by delivering to the site of implantation the small molecule inhibitor YM155. The YM155 can be encapsulated in a sustained-release encapsulating agent.

In a sixth aspect the present invention provides a method of inhibiting or reducing the FBR responsive to the implantation of a medical device by delivering to the site of implantation a small molecule inhibitor of survivin. In an advantageous embodiment the small molecule inhibitor of survivin is YM155, FL118, SF002-96-1, Terameprocol, WM-127, GDP366, Abbot 8, LLP3, LLP9, S12, Indinavir, Nelfinavir, LQZ-7, LQZ-7F, LQZ-7I, Shepherdin, AICAR, Deazaflavin analog compound 1, UC-112, MX-106, Compound 12b, Compound 10f, Compound 10h, Compound 10k, Compound 10n, PZ-6-QN or combinations thereof. The small molecule inhibitor is advantageously encapsulated in a sustained-release encapsulating agent.

In a seventh aspect the present invention provides a system to screen for cFLIP inhibitors. The method employs a macrophage cell line that overexpresses cFLIP such as that disclosed herein. The method for screening cFLIP inhibitors can include the step of contacting the macrophage cell line with one or more compounds to be tested for cFLIP inhibition. The compound to be screened can include YM155, FL118, SF002-96-1, Terameprocol, WM-127, GDP366, Abbot 8, LLP3, LLP9, S12, Indinavir, Nelfinavir, LQZ-7, LQZ-7F, LQZ-7I, Shepherdin, AICAR, Deazaflavin analog compound 1, UC-112, MX-106, Compound 12b, Compound 10f, Compound 10h, Compound 10k, Compound 10n, PZ-6-QN and combinations thereof.

### BRIEF DESCRIPTION OF THE DRAWINGS

For a fuller understanding of the invention, reference should be made to the following detailed description, taken in connection with the accompanying drawings, in which:

FIG. 1 is an illustration depicting that cFLIP asserts prosurvival mechanisms by inhibiting caspase-8 mediated apoptosis.

FIG. 2 is an illustration depicting that a novel triple transgenic system (cFLIP^{Δ/Δ}) that conditionally deletes CFLAR (the gene that encodes cFLIP), but only in DOX-induced hCD68-rtTA responsive cells.

FIG. 3 is a set of graphs and images labeled (a)-(c). (a) DOX activation of hCD68-rtTA in blood monocytes and tissue-resident macrophages in naïve lung shown with a GFP reporter. (b) DOX administration to cFLIP^{Δ/Δ} mice promotes macrophage apoptosis. Assessed by TUNEL staining after bleomycin treatment at the indicated time. (c) DOX administration to cFLIP^{Δ/Δ} mice inhibited bleomycin-induced lung fibrosis as assessed histologically and by hydroxyproline release.

FIG. 4 is an illustration depicting the design and endpoint analyses for monocyte-derived “recruited” (Rec) and tissue-resident (Res) macrophages.

FIG. 5 is a set of illustrations labeled (a) and (b) depicting endpoints for flow and IHC analysis.

FIG. 6 is a set of illustrations labeled (a) and (b). (a) Photolabile microparticle fabrication and (b) Light-induced microparticle degradation and YM155 release.

FIG. 7 is an illustration depicting endpoints for flow and IHC analysis.

FIG. 8 is an illustration depicting the foreign body response that occurs as a result of the innate immune system responding to foreign material being inserted into a body.

FIG. 9 is an illustration depicting the caspase-mediated extrinsic pathway.

FIG. 10 is a schematic of cre-driven transgenic murine model that is initiated by tamoxifen treatment and deletes the gene CFLAR from macrophages.

FIG. 11 provides two illustrations depicting in vitro assessment of cell behavior. (A) In Vitro Experimental Protocol: J774A.1 macrophage cell line was incubated overnight at 20,000 cells/well and treated with lipopolysaccharide (LPS) for 4 hours or 3days. Exogenous FasL was then added to initiate the extrinsic apoptotic pathway for 6 hours before caspase activity (Caspase-Glo3/7) and metabolic activity (alamarBlue) assays were performed to study the differences in cell behavior after exposure to each stimulant. (B) In Vitro Experimental Protocol for Small Molecule Testing. J774A. 1 macrophage cell line was incubated overnight at 20,000 cells/well and cultured incomplete media for 3 days. Exogenous FasL was then added to initiate the extrinsic apoptotic pathway with or without a small molecule inhibitor for 6 hours before a caspase activity (Caspase-Glo8) assay was performed to study the differences in cell behavior after exposure.

FIG. 12 is eight graphs (labeled (A)-(H)) depicting cell identification via fluorescent staining. Gating strategy for distinguishing immune cell types from the surrounding implant tissue area. Flow cytometry was used to distinguish immune cell types: CD45+ leukocytes, CD11b+ myeloid cells, Ly6G+ neutrophils, Ly6C+ monocytes, F4/80+macrophages, and Zombie Dye to distinguish dead cells. tdTomato positivity was achieved via tamoxifen injections given to the reporter transgenic mouse model.

FIG. 13 is three sets of graphs (labeled (A)-(C)) depicting that macrophage frequency is higher relative to other myeloid cells, and nearly all monocytes and macrophages are tdTomato positive with tamoxifen (TMX) treatment. (A) Temporal frequency of neutrophils (N), monocytes (Mo), and macrophages (Mac). (B) Nearly all macrophages and monocytes are tdTomato⁺ with continual TMX treatment. (C) tdTomato⁺ macrophages and monocytes decrease over time, but by day 29 over half of the macrophages and monocytes are persistent; TMX treatment up to day 7. *p<0.05; **p<0.01; ***p<0.001; symbol above a column compares to day 1.

FIG. 14 is an illustration (A) and two sets of images ((B) and (C)) showing a cFLIP conditional knockout model that qualitatively indicates a looser fibrous capsule at 28 days post-implantation with tamoxifen treatment. (A) Schematic of implant surgeries on mice (B) Implant and histology samples from cFLIP knockout model (C) Implant and histology sample from littermate control model; Fibrous capsule above black star; black star indicates implant site.

FIG. 15 provides quantification of capsule thickness on day 29 post-implantation. The capsule thickness was lower (p=0.08) in cFLIP^{Δ/Δ} mice, which is consistent with the histology images, compared to the littermate control.

FIG. 16 is a set of three graphs ((A)-(C)) and a set of images (D) of an in vitro model to recapitulate cell persistence using a J774A.1 cell line sensitive to stimuli causing pro-survival behavior. Caspase production was significantly decreased with long-term exposure to an inflammatory stimulant which will allow for testing small molecules to reverse the induced pro-survival behavior in future experimentation. (A) compares the fold-change in caspase 3/7 activity between each condition relative to untreated control conditions; ABCDE signifies p<0.05 between indicated FasL concentrations; D*E* indicates significant differences from both FasL Only and LPS, FasL conditions. (B) indicates metabolic activity using an alamarBlue assay of each tested condition relative to untreated control conditions. (C) shows differences in caspase 3/7 activity when cells were stimulated with LPS for either 4 hours or 3 days then subjected to FasL stimulation at a concentration of 100 ng/ml for 6 hours. (D) shows qualitative images of each well plate after 3 days in varied concentrations of LPS. N=3; *p<0.05.

FIG. 17 is a set of two graphs (labeled (A) and (B)) demonstrating studies utilizing a small molecule inhibitor of CFIP where the small molecule (YM155) exposure leads to increased caspase 8 activity. (A) Cells treated with 100 ng/mL of small molecule expressed a fold change of about 23 times greater than the untreated control. (B) Confirmation that the small molecule is causing increased caspase activity rather than the solvent it is dissolved in. N=3

FIG. 18 is a set of 18 drawings providing the structure of the survivin small molecule inhibitors listed in Table 1. From: Albadari N, Li W. Survivin Small Molecules Inhibitors: Recent Advances and Challenges. Molecules. 2023 Feb. 1; 28(3):1376. doi: 10.3390/molecules28031376. PMID: 36771042; PMCID: PMC9919791.

### DETAILED DESCRIPTION OF THE PREFERRED EMBODIMENT

Macrophages are key players in the foreign body response (FBR) to implanted biomaterials, in which an avascular fibrous capsule walls off the implant from the surrounding tissue. While the concept that macrophages are required for the FBR is well-accepted, the cellular mechanisms that contribute to the FBR have not been elucidated. Research efforts have largely focused on the transition from pro-inflammatory to wound-healing macrophages as the driver of fibrosis. The latter is complicated by the diverse roles of wound-healing macrophages. As a result, successes with synthetic-based biomaterials have been limited to ones that the body tolerates and which function despite a FBR. However, to fully achieve integration of the biomaterial to the host tissue (e.g., vasculature), strategies that mitigate fibrous encapsulation are needed.

In tissue fibrosis, macrophages upregulate pro-survival (i.e., anti-apoptotic) molecules that lead to macrophage persistence. However, if these pro-survival mechanisms are inhibited, macrophages can be resensitized to apoptotic cell death, and tissue fibrosis can be prevented or resolved. Cellular FLICE-like inhibitory protein (cFLIP) is a major regulator of macrophage cell fate. Inhibition of cFLIP in macrophages can prevent tissue fibrosis. There are parallels between tissue fibrosis and the FBR, both of which are types of impaired wound healing. We hypothesized that macrophage persistence in the FBR is mediated by intracellular cFLIP. Thus, inhibiting cFLIP will resensitize macrophages to apoptotic death signals to prevent or resolve fibrous encapsulation.

A mouse model was developed that uses the hCD68-rtTA transgenic system to conditionally deplete cFLIP in myeloid cells. This innovative mouse model, combined with advanced immunological methods, enables the elucidation of the cellular mechanisms involved in fibrous capsule formation of the FBR.

The foreign body response (FBR) is a formidable response that occurs to all implantable biomaterials. The FBR is a chronic inflammatory response that leads to the walling off of the implantable medical device with a fibrous capsule. This fibrous capsule is a dense matrix that blocks communication and integration with the surrounding tissue. Macrophages are the innate immune cells responsible for the FBR. We have identified a pathway in macrophages, which causes their resistance to apoptosis, and as a result leads to their prolonged presence at an implant. The data presented herein indicates that inhibiting this pathway causes macrophages to undergo apoptosis and surprisingly appears to completely abrogate formation of the fibrous capsule. A target in the immune cell has been identified that is responsible for the FBR. By tethering a small molecule to a biomaterial that inhibits this pathway, the FBR can be prevented.

The foreign body response (FBR) is driven by innate immune system responses to implanted non-biological biomaterials and is characterized by formation of an avascular fibrous capsule. Success of current FDA-approved implantable medical devices relies on the body's ability to tolerate them. However, scaffolds for tissue engineering and other devices, such as glucose sensors, that require integration into the host tissue and its vasculature need material designs that prevent fibrous encapsulation.

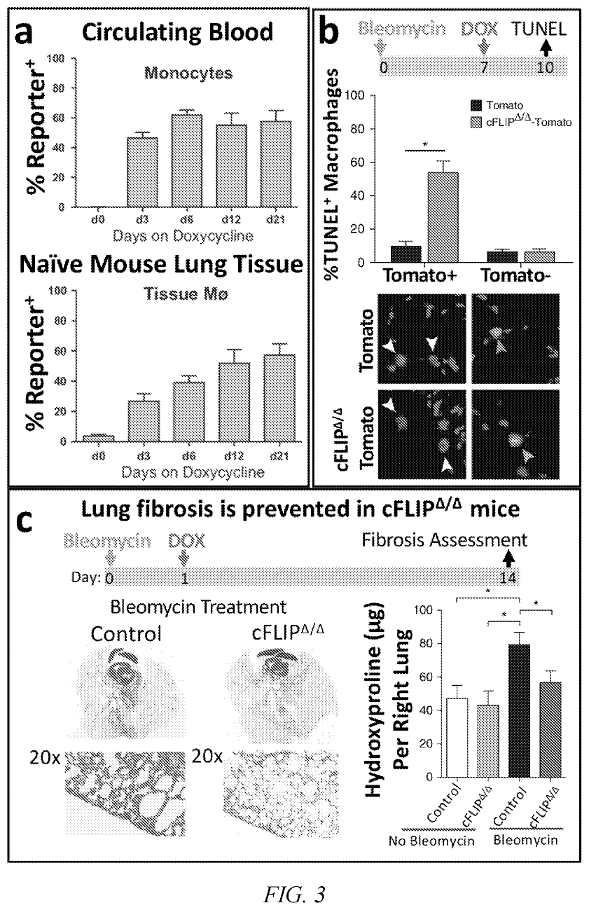

Under normal circumstances, wound healing follows a highly coordinated series of events that include: a) influx of inflammatory cells to the injury site, b) proliferation of structural cells and formation of a provisional matrix, c) maturation of granulation tissue, and d) matrix remodeling with return to homeostasis. Macrophages are essential to normal wound healing. During early phases they exist in high numbers, removing debris and dying cells and orchestrating fibroblast proliferation and collagen production. Once the tissues have been repaired, macrophages undergo apoptosis, and collagen production ceases. Tissue fibrosis represents pathologic wound healing and is characterized by excessive accumulation of matrix proteins (including collagen). In this context, macrophages are key drivers of tissue fibrosis and that appropriately timed deletion of macrophages attenuates fibrosis and improves organ function (see FIG. 3).

The FBR can be considered an impaired wound healing response. The hallmarks of the FBR are accumulation of macrophages on and around the implant and the formation of a fibrous capsule-both of which persist for the lifetime of the implant. These same features represent the cardinal hallmarks of tissue fibrosis. Accordingly, many of the mechanisms that drive fibrosis are responsible for the development of the FBR. A key mechanism that underlies macrophage persistence in fibrotic tissues is resistance to apoptosis. Therefore, strategies that re-sensitize macrophages to undergo apoptosis represent a novel therapeutic approach to limit the FBR.

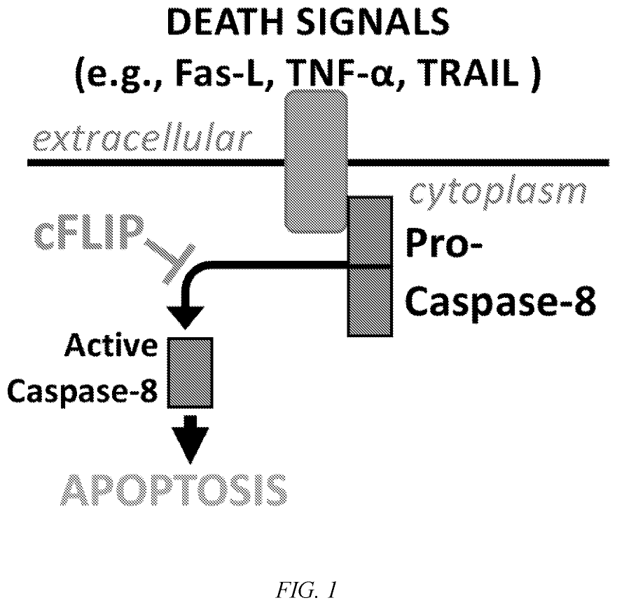

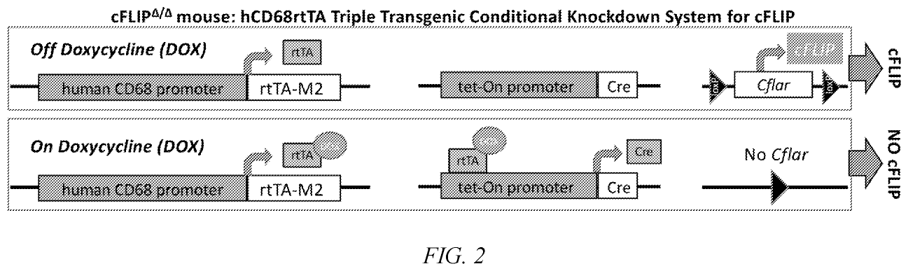

Cellular FLICE-like inhibitory protein (cFLIP) is a central determinant of cell fate (FIG. 1). When a cell receives a death signal from the extracellular environment, caspase-8 is activated and apoptosis is triggered. However, when cFLIP is present, caspase-8 signaling is blocked and the cell evades death. As an example, high expression of cFLIP in cancer cells blocks their cell death. To address the role of cFLIP in tissue fibrosis, a novel triple transgenic mouse model was developed that can be used to inducibly delete cFLIP in macrophages (FIG. 2). The mice are designated cFLIP^{Δ/Δ}. This system works as follows: Reverse tetracycline-controlled transactivator (rtTA) is constitutively expressed in cells using the CD68 promoter (which drives expression in myeloid cells including macrophages but not non-myeloid cell types). When mice are fed doxycycline (DOX), DOX binds to and activates rtTA molecules. The rtTA-DOX complex activates the tetracycline response element (TRE) and drives expression of Cre recombinase, which deletes Cflar (the gene that encodes cFLIP). With DOX, Cflar is deleted and cFLIP is no longer produced. Without DOX, Cflar is transcribed normally (FIG. 2).

The cFLIP^{Δ/Δ} system can resensitize macrophages to apoptosis and prevent bleomycin-induced lung fibrosis. The hCD68-rtTA system is activated in blood monocytes and in tissue resident lung macrophages (FIG. 3(a)). DOX administration after bleomycin resensitizes lung macrophages to death signals, leading to their apoptosis (FIG. 3b), and attenuated fibrosis (FIG. 3c). The DOX administration can be timed to re-sensitize macrophages to apoptosis at distinct time points during the inflammatory response.

Macrophages persist in and are required for the FBR, but the cellular mechanisms remain unclear. Studies in tissue fibrosis have shown that resistance to apoptotic death signals leads to macrophage persistence and tissue fibrosis. Lung fibrosis models shows that macrophages can be re-sensitized to apoptotic cell death signals through deletion of cFLIP. Sensitization of macrophages to apoptosis can prevent development of fibrosis (as in FIG. 3) or can hasten the reversal of established fibrosis. The similarities between tissue fibrosis and the FBR provide a compelling scientific basis that ‘pro-survival’ macrophage programming is a key driver of the FBR that can be reversed through inhibition of cFLIP.

Identifying pro-survival macrophage programming opens doors for new targets to prevent the FBR. While other pathways are almost certainly involved in creating the FBR, targeting ones that block macrophage recruitment or inhibit macrophage function may disrupt tissue healing and integration with the implant and may, under certain circumstances, increase the risk for device-associated infection. Accordingly, appropriately timed inhibition of cFLIP will enable macrophages to perform critical early immune functions and tissue functions, but render them sensitive to later apoptotic death signals.

Macrophages in the FBR adopt pro-survival programming that leads to and maintains the fibrous capsule. This survival mechanism emerges due to the presence of intracellular cFLIP, which renders macrophages refractory to death receptor signaling. Conversely, cFLIP depletion in macrophages can re-sensitize them to apoptosis when death signals are present. The innovative mouse models we have developed can be utilized to elucidate these mechanisms. Under DOX control, targeted cells are conditionally induced to express the fluorescent tdTomato reporter alone (hCD68rtTA-tdTomato mice) or with simultaneous depletion of cFLIP (cFLIP^{Δ/Δ}-tdTomato). The former permanently labels targeted cells and enables lineage tracing. The latter induces resensitization of targeted cells to apoptotic cell death.

Multiparameter flow cytometry can be combined with lineage tracing using hCD68rtTA-tdTomato mice to identify subsets of myeloid cells in the subdermal layer of skin and determine a) how long each population persists and b) changes in expression of fibrosis-relevant genes in each population in time in the FBR to subcutaneous implants. Tissues including dermis contain multiple subtypes of macrophages; at least five unique populations of dermal mononuclear phagocytes have been identified.

Advanced biomaterial designs can be applied to determine optimal timing to resensitize macrophages to apoptosis using a therapeutic drug for preventing the FBR. A pharmacological inducible system based on phototriggerable biomaterials can be designed that upon light exposure induces local release of YM155. YM155 is a small molecule that is chosen because: a) it inhibited cFLIP in cancer cells and induced their death without affecting non-cancerous cells, and b) it reversed anti-apoptotic programming in macrophages in vitro.

Through these innovations, the kinetics of macrophage recruitment and survival can be elucidated, fibrotic tissue remodeling factors in macrophages can be characterized as they start to persist at the implant, and the role of cFLIP in the FBR can be determined. Therapeutically, optimal timing for depleting cFLIP in the FBR can be identified (in mouse models), and pharmacologic cFLIP inhibition can be demonstrated, such as via YM155 release.

It is submitted that: (a) tissue fibrosis and the FBR are in many ways similar and (b) the presence of cFLIP in macrophages prevents their death and contributes to tissue fibrosis. (1) Macrophages in the FBR adopt pro-survival programming during the transition from inflammation to fibrosis; and (2) Expression of cFLIP is responsible for macrophage persistence in the FBR, and when inhibited, macrophages are resensitized to apoptosis and the fibrous capsule is prevented or resolved depending on timing. Therapeutic biomaterials can be investigated using systems taught herein with on-demand YM155 release for functional improvements in implant performance.

### Example 1: The Kinetics of Macrophage Persistence in the FBR to Distinct Implants is Elucidated

Studies on (a) the kinetics of recruitment and survival of macrophage subsets during the FBR and (b) changes that occur in the expression of fibrotic tissue remodeling factors in macrophage subsets and over time are performed to investigate the FBR as shown below.

Lineage tracing experiments are performed in hCD68rtTA-tdTomato mice and when combined with multiparameter flow cytometry, “recruited” (i.e., blood-monocyte derived) and tissue-resident macrophages are identified and distinguished by their temporal patterns in the FBR and changes in their fibrotic gene expression.

Four synthetic-based polymeric implants that vary in chemistry and stiffness: two PEG hydrogels, silicone, and polyetheretherketone (PEEK), are chosen for their wide use in tissue engineering and permanent medical devices. Accordingly, temporal differences in macrophages to distinct implants can be identified.

c-FLIP can be temporally-inhibited in macrophages to promote their programmed cell death and attenuate formation and maintenance of the fibrous capsule in the FBR. The inhibition of cFLIP in macrophages promotes their programmed cell death and attenuates formation and maintenance of the fibrous capsule in the FBR.

cFLIP inhibition in macrophages can be used to determine the temporal effects on the FBR. A hCD68-rtTA transgenic mouse coupled with a tet-On Cre system that deletes cFLIP can be utilized. This system targets myeloid cells and can be temporally controlled by administration of doxycycline to delete the gene that encodes cFLIP, as shown below. This mouse model elucidates the temporal effects of cFLIP deletion in myeloid cells on the formation of the fibrous capsule and on its dissolution.

A phototriggerable biomaterial can be used to inhibit cFLIP temporally and locally in macrophages. Photo-labile microparticles are embedded within a biomaterial, which when triggered by light lead to the slow release of YM155, a small molecule inhibitor of cFLIP. By tightly controlling the release of YM155, the temporal and local effects of cFLIP inhibition by a biomaterials strategy are determined.

A system is provided that enables: (a) determination of the temporal patterns of macrophage accumulation and their persistence in the FBR, (b) elucidation of the role of cFLIP in mediating long-term survival of macrophages and its effect on fibrous encapsulation, (c) identification of the optimal timing for depleting cFLIP, and (d) the development of strategies for preventing and/or resolving the FBR. The present invention thus enables methods and systems for the long-term prevention of the FBR and concomitant functional improvement in the performance of implantable biomaterials.

Biological Variables and Scientific Rigor. Animals: hCD68rtTA-tdTomato mice can be used, along with cFLIPA/A-tdTomato mice, both of which are on the C57BL/6 background. The former has the same driver depicted in FIG. 2, but rather than floxed cFLIP, express a floxed stop codon. Accordingly, cells that activate hCD68 (and their progeny) will permanently express Tomato. Breeding pairs can be used to generate a sufficient number of mice for this project, and wildtype C57BL/6 mice can be purchased and/or bred as needed. Mice aged between 6-8 weeks can be used. Male and female mice can be used and sex-disaggregated data reported.

Statistical analysis: Assumptions of parametric data can be tested using Shapiro-Wilk test for normality of data distribution and Levene's test for homogeneity of variance. For parametric data, one-way or two-way ANOVA and Tukey's post hoc analysis can be used. For non-parametric data, a Kruskal-Wallis test can be used with a post hoc pairwise Mann-Whitney U with a Bonferroni correction. Significant differences between the groups can be determined at the level of a=0.05.

Power analyses (a=0.05; b=0.2) were run to determine sample size. Animal studies can use a n=6, based an 60% difference in fibrous capsule thickness quantified from immunohistochemistry analysis. Power analysis for flow cytometry/FACS assays can be re-run and adjusted for sample size. In vitro studies in Aim 2.2 can use n=3, based a 75% difference in the primary readout (caspase-8 activity) in apoptotic macrophages.

The kinetics of macrophage persistence in the FBR to distinct implants can be investigated using systems taught herein. Macrophages are a hallmark of the FBR and are known to promote fibrous encapsulation, but their origin is unclear. Lineage tracing experiments can be performed in hCD68rtTA-tdTomato mice with distinct implants to determine a) the kinetics of recruitment and survival of macrophage subsets in the FBR and b) changes in expression of fibrotic tissue remodeling factors by macrophage subsets over time.

Overall Experimental Design. Four synthetic-based polymeric biomaterials that range in chemistry and stiffness can be tested: PEG hydrogels of two stiffnesses, silicone, and polyetheretherketone (PEEK). The FBR to PEG hydrogels for their potential in tissue engineering and as coatings for implants has been studied. We have shown a greater FBR to PEG hydrogels with increased stiffness, which implicates increased involvement of the systems to reduce or eliminate the FBR as taught herein.

Silicone is widely used in medical devices and its FBR is well-characterized. PEEK, a stiff biomaterial (Young's modulus, 3-4 GPa), is used in many current medical devices, e.g., in orthopedics and cardiology. The subcutaneous site can be chosen for implantation because it has been studied the FBR at this site extensively, but the approach taught herein can be applied to any tissue. As the FBR is an ubiquitous response, accumulation of myeloid cells should be similar across the implants, but the timing when macrophages become persistent will vary. Temporal differences can be identified to distinct implant types.

Naïve mice that are fed DOX chow for 7 days continuously can be examined, which is sufficient time to turn on the reporter. At day 7, the fraction of Tomato+ myeloid cell subsets in the subdermis can be determined by flow cytometry and spatial location of Tomato+ macrophages will be confirmed by histology. This experiment can be used to confirm the myeloid cell subsets that activate hCD68 in the subdermis and the timing for DOX.

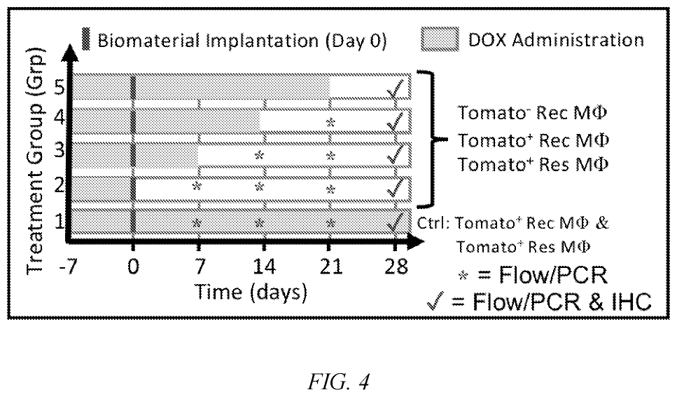

Pulse-wait experiments can be applied (FIG. 4). These distinguish between recently arrived and persistent (i.e., prosurviving) macrophages. During the pulse period (i.e., DOX administration), myeloid cells will become Tomato+. Upon withdrawal, blood monocytes will be replenished from stem cells in the bone marrow within ˜2 days resulting in Tomato− monocytes. Monocyte-derived macrophages (referred to as “recruited”) arriving at the implant at this time will also be Tomato−. Thus, Tomato+ “recruited” macrophages observed after DOX withdrawal will indicate persistent cells. By varying the timing when DOX is withdrawn (Grp #2-5, FIG. 4), the point in the FBR at which macrophages adopt prosurvival programming can be determined. Tissue-resident macrophages can also be labeled during the pulse period, but will remain Tomato+ due to origin and their self-renewal capacity. Using flow cytometry and FACS, one can distinguish between and characterize the fibrotic tissue remodeling factors of each macrophage subset over time.

Biomaterial Implantation. Medical-grade silicone (Invotec) and PEEK (90G, Victrek) can be obtained. Two PEG hydrogels (G, shear modulus) can be fabricated: G=10 kPa for soft tissue engineering and G=75 kPa for hard (e.g., bone) tissue engineering. PEG hydrogels can be formed from thiol and norbornene multi-arm macromers purchased or synthesized. Macromer molecular weight and concentration can be varied to tune modulus. Stiffness-matched silicone (G=75 kPa) can be chosen. Biomaterial disks (5 mm diameter, 1 mm thick) can be sterilized, confirmed endotoxin-free and placed in separate subcutaneous pockets. Each mouse can receive 4 implants; one of each type, placed on the right and left side and over shoulders and hips and for up to 28 days, which spans the timing for fibrous encapsulation (˜14 days).

Identification of Tomato+ and Tomato− myeloid cell subsets by flow cytometry (Flow). 1 cm² sections of subdermal skin in naïve mice can be collected. Grp #1-5: Biomaterials and immediate surrounding tissue can be explanted.

All specimens can be enzymatically digested to liberate cells. Isolated cells can be fixed and Fc receptors blocked with anti-CD16/32 monoclonal antibody. Cells can be stained with antibodies directed at CD45, CD11b, CD3, CD19, Ly6G, Siglec-F, Ly6C, CD11c, CCR2, CD64, and MHCII. Following exclusion of doublets and dead cells (DAPI+), CD45− tissue cells, eosinophils (Ly6G−, Siglec-F+) and lymphocytes (CD3+,CD19+) can be identified and confirmation can be made that they are Tomato− (i.e., do not activate hCD68-rtTA). Subsets of myeloid cells can be identified as follows: neutrophils (Ly6G+, Siglec-F−, SSClo), monocytes (Ly6G−, Ly6C+, Siglec-F−, SSClo, CD64lo), monocyte-derived macrophages (Ly6G−, Ly6C−, CD64lo, CCR2+, MHCII+), tissue-resident macrophages (Ly6G−, Ly6C−, CD64hi, CCR2−, MHCIIlo-hi). Multinucleated foreign body giant cells (FBGCs) can be identified by their high forward and side-scatter, propidium iodide staining for polyploidy and co-expression of macrophage markers (Ly6G−, Ly6C−, CD64lo-hi, MHCIIlo-hi). One can then quantify percent Tomato+ and Tomato− cells for each myeloid cell subset and time point. Animal numbers: 15 endpoints (Flow)×6 mice (replicates)=90 mice (FIG. 4).

Characterization of macrophages (qPCR). Grp #1-5 (FIG. 4): Tomato+ and Tomato− “recruited” macrophages and tissue-resident macrophages can be isolated by fluorescence activated cell sorting. RNA can be isolated and quantitative RT-PCR performed to assess fibrotic tissue remodeling factors of each macrophage subset at each time point. One can then measure inflammatory cytokines (e.g., Il1b, Tnfa, Il6) and chemokines (e.g., Cxcl1, Ccl2, Ccl5), tissue growth factors (e.g. Vegfa, Hgf, Pdgfb) associated with fibrosis, and a stable housekeeping gene (Rpl32). Animal numbers: 14 endpoints (qPCR)×6 mice (replicates)=84 mice (FIG. 4).

Spatial identification of macrophages by immunohistochemistry (IHC). 1 cm2 full-thickness skin from backs of naïve mice will be removed from the same mice used in flow cytometry. Grp #1-5: Biomaterials and immediate surrounding tissue will be explanted at day 28. Both will be fixed, embedded, and cryosectioned. MerTK antibody will be used to stain all macrophages, with DAPI counterstain to identify all cells. Tomato+ macrophages will be identified. Animal numbers: 5 endpoints (IHC)×6 mice (replicates)=30 mice (FIG. 4).

At days 7 and 14 post-implantation, Tomato+ “recruited” macrophages will emerge after DOX withdrawal and be present at the later endpoints (Grp #3-4). Newly recruited macrophages will highly express pro-inflammatory cytokines and chemokines while persistent macrophages will preferentially express fibrotic tissue growth factors. The origin of FBGCs is not well-established. Tomato− FBGCs in Grp #2-3, but not Grp #1,4-5 will indicate a monocyte-derived macrophage origin. If mixed Tomato+/− cells persist in this group, then it will suggest a resident and recruited origin for FBGCs.

At homeostasis in skin a small fraction of tissue-resident macrophages are repopulated by blood monocytes. While neutrophils will activate hCD68-rtTA, they are short-lived and they are not required for fibrous capsule formation. Transgenic systems do not always behave as expected and thus a reporter studies can be used to test the fidelity of the system in skin. If necessary, a tamoxifen-inducible CX3CR1-CreER system can be as an alternative to CD68-rtTA. In case of mixed Tomato+/− in Grp #3-4, a treatment group can be added to clarify origin: 7-day DOX followed by 7-day withdrawal prior to implantation such that no circulating monocytes are Tomato+.

As shown in further examples below, cFLIP was temporally inhibited in macrophages to promote their programmed cell death and attenuate formation and maintenance of the fibrous capsule in the FBR. cFLIP imparts pro-survival programming to macrophages that contributes to their persistence and that leads to formation and maintenance of the fibrous capsule. cFLIP inhibition resensitizes macrophages to death signals, leads to their apoptosis when death signals are present, and reverses fibrosis. The inducible cFLIP^{Δ/Δ} mice can be used to study the temporal role of cFLIP inhibition in the FBR and to determine the time at which cFLIP deletion is protective for each biomaterial. A pharmacological inducible system has been developed using a phototriggerable biomaterial that releases YM155 on-demand to investigate the temporal role of local cFLIP inhibition on fibrous capsule formation and resolution.

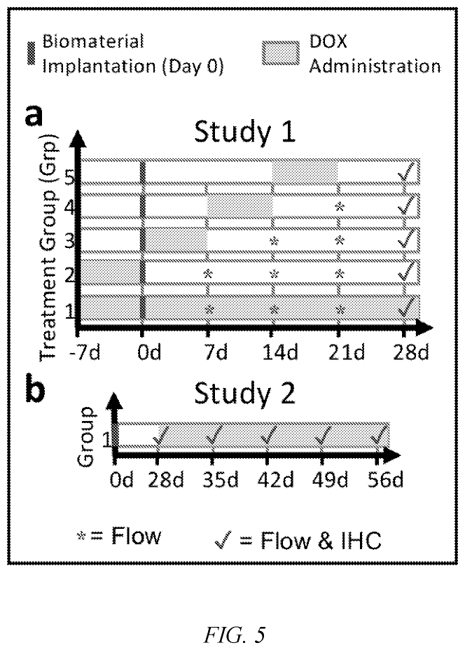

Overall Experimental Design. Two studies are performed to test the effect of cFLIP depletion on fibrous capsule formation and to show that depletion of cFLIP at later time points can resolve fibrosis around the implant. The four biomaterials are implanted in cFLIP^{Δ/Δ}-tdTomato mice and hCD68rtTAtdTomato control mice. DOX can be administered for the full time course or at selected intervals (FIG. 5a). DOX can be administered after the fibrous capsule has formed (Ñ28 d) (FIG. 5b). Both studies can quantify percent Tomato+ myeloid cells (Flow), Tomato+ apoptotic macrophages (i.e., Annexin V, TUNEL) (Flow & IHC), and total macrophages (IHC), and also fibrous capsule thickness (IHC).

Biomaterial Implantation & FBR Assessment. Biomaterials can be implanted in cFLIP^{Δ/Δ}-tdTomato and in control mice that do not have floxed Cflar and therefore retain intact cFLIP levels. At each endpoint, specimens will be analyzed by Flow and IHC (see Aim 1). Animal Numbers: Study 1: [14 (Flow)+5 (IHC)]=19 endpoint analyses; Study 2: [5 (Flow)+5 (IHC)]=10 endpoint analyses. Total=29 endpoint analyses×2 genotypes×6 mice (replicates)=348 mice.

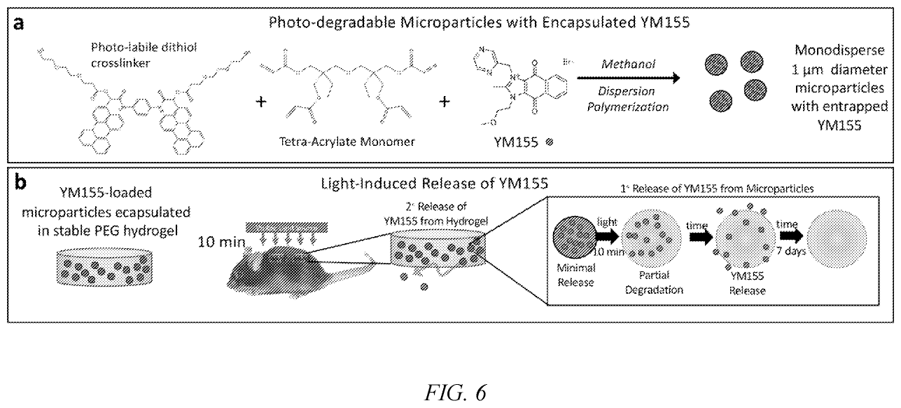

A pharmacological strategy can be used to locally inhibit cFLIP in cells surrounding the implant at discrete times in the FBR. A biomaterial can be designed to release YM155 (or another small molecule inhibitor of cFLIP) on-demand by exposure to light. photolabile YM155-loaded microparticles can be fabricated (FIG. 6a) and encapsulated in a PEG hydrogel (G=75 kPa). YM155 will remain localized to high crosslinked microparticles until the extent of light-activated particle degradation reaches a particle mesh size that is greater than YM155. Once released from the particle, YM155 will rapidly diffuse (˜2 hr) out of the PEG hydrogel due to YM155's small size (FIG. 6b). Thus, YM155 release to the in vivo environment is controlled by its release from the particles.

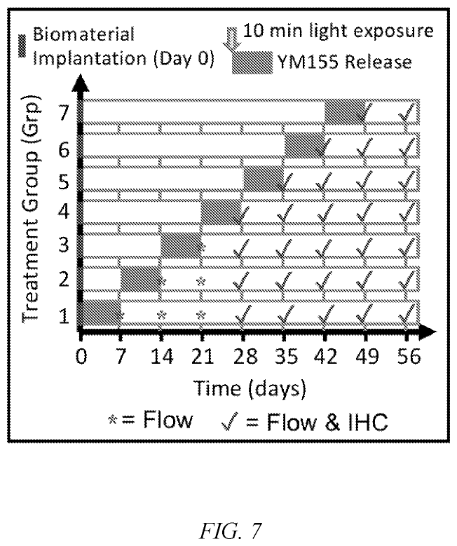

To induce microparticle degradation and YM155 release following biomaterial implantation in wildtype mice, animals will be placed under a visible lamp (e.g., similar high intensity to dental lamps) at prescribed times (FIG. 7). Because visible light penetrates skin (e.g., 50% transmission of 550 nm light through 0.5 mm thick skin), degradation will be controlled by energy dose (i.e., time and intensity of light). YM155 release over a discrete time frame (e.g., 7 days) will be targeted, which is expected to be sufficient to affect macrophages that surround the implant, but which can be extended based on results. The efficacy of this system is shown in vitro in macrophages and in vivo in subcutaneous implants.

Biomaterial Design. Monodisperse crosslinked microparticles can be prepared from thiol-Michael addition dispersion polymerization. Photolabile diacrylate monomers can be synthesized with perylene as a visible light responsive chromophore because of its demonstrated cytocompatibility and when incorporated into the crosslinks of a hydrogel led to its rapid degradation by 530 nm light. The Passerini multicomponent reaction scheme can be used, where thiol-PEG2-acid (BroadPharm) is dissolved in methylene chloride and perylene-3-carbaldehyde (Tokyo Chemical Industry) is added at 1:1 moles of aldehyde: carboxyl groups followed by addition of 1,4-phenylene diisocyanide (Sigma) at a 1:1 moles of socyanide: carboxyl groups and then stirred for 20 hours at room temperature. The final dithiol crosslinker (FIG. 6a) can be purified by dialysis, lyophilized, and confirmed by 1H NMR. Microparticles loaded with YM155 (Cayman) will be formed by reacting the photolabile dithiol and di(trimethylolpropane) tetraacrylate (DTPTA) at stoichiometric ratios of thiol to ene in methanol with polyvinylpyrrolidine as a surfactant and with YM155 for 2 hours. The reaction can be initiated by the catalyst hexylamine, whose concentration controls microparticle size. One can target 1 um diameter using 10% (g/g) catalyst and confirm size by scanning electron microscopy.

Ex Vivo Characterization of YM155 release. Characterization of degradation kinetics of microparticles can be shown under 530 nm light. One can characterize release using a model drug of similar molecular weight to YM155, the fluorophore Alexa Fluor 350. One can quantify by fluorescence, loading efficiency and release kinetics in microparticles and after encapsulation in the hydrogel as a function of energy dose. One can target two million particles/hydrogel or <1% (v/v) and a loading of 1 μg YM155 per gel. This concentration is lower than that which has been tested in subcutaneous injections for tumor studies in mice, but higher than that which has been used in in vitro cultures with macrophages. One can target a less than or equal to 10 minute exposure and adjust particle concentration, drug loading, and polymer crosslink density to achieve sustained release of the model drug over one week after light exposure. One can determine light transmittance through mouse skin and use that light intensity to study release kinetics in vitro. One can also confirm YM155 loading efficiency into and biological activity after release from microparticles in vitro. One can isolate bone-marrow monocytes from cFLIP^{Δ/Δ} and control mice and differentiate them into macrophages in vitro (2×106 macrophages/mouse).

Macrophages can be cultured in 2D with transwells holding the microparticle-loaded hydrogels. Macrophages can be treated +/− Fas-L (a death signal) and hydrogels +/− YM155-particles and +/− light exposure. Macrophages +/− DOX and +/− Fas-L can serve as positive controls for cFLIP inhibition. Follow-up studies can be performed by seeding macrophages directly on hydrogels. One can assess death-receptor apoptotic signaling by caspase 8 activity. These experiments will validate this system for YM155 release. Animal Numbers: 12 mice.

Biomaterial Implantation and FBR Assessment. two hydrogels per mouse can be implanted; one between the shoulders and one between the hips. Each treatment (FIG. 7) can have YM155-microparticles and microparticles-only in hydrogels. At prescribed times, mice can be anesthetized, their backs shaved, and each implant exposed to light using a light guide equipped with a 530 nm bandpass filter that is connected to a collimated LED for 10 minutes and then returned to their cages. Analysis can be performed as described above. In addition, bystander TUNEL+ fibroblastic (a-smooth muscle actin+) and non-fibroblastic cells can be quantified (MerTK-a-smooth muscle actin-) that may be impacted by YM155. Animal Numbers: [35 (flow)+29 (IHC)]=64 endpoint analyses×2 materials×6 mice (replicates)=768 maximum mice.

Optimal timing of cFLIP depletion or inhibition is expected to be between days 7-14. When macrophages are able to undergo apoptotic cell death, fibrous encapsulation will be prevented. cFLIP depletion in cFLIP^{Δ/Δ} mice or inhibition by YM155 release after fibrous encapsulation will lead to dissolution of the fibrous capsule over time. Comparing the pharmacological studies to the cFLIP^{Δ/Δ} mouse studies allows assessment of cFLIP expression in fibroblast cells and their persistence influences on formation of the fibrous capsule. Potential pitfalls and alternative solutions.

Inefficient diffusion of DOX across the fibrous capsule once its formed can be confirmed if control mice have limited Tomato− macrophages at the implant. DOX treatment can be extended >7 days, but if macrophages remain Tomato−, it may implicate that the fibrous capsule is too dense and that local delivery from the implant will be required. Hydrogels have been successfully photopolymerized under the skin of a mouse and hydrogels with perylene crosslinks completely degrade in <10 minutes by 530 nm light at 10 mW/cm², which suggests the efficacy of photodegrading the microparticles in vivo. Although our microparticles are more highly crosslinked, only partial degradation is required to achieve a mesh size that allows YM155 release. Should release be too rapid, particle hydrophobicity can be increased to reduce mesh size and slow release in aqueous environments. YM155 can be immobilized to the surface of silicone and PEEK implants with the same light-sensitive linker to assess cFLIP inhibition and the FBR.

### Example 2: Apoptotic Pathways in the Immune Response to Implanted Biomaterials

The foreign body response (FBR) occurs as a result of the innate immune system responding to a foreign material being inserted into the body. Macrophages are immune cells that play a key role in the progression of the FBR. Frustrated macrophages fuse together to form foreign body giant cells. Macrophages signal fibroblasts to encapsulate the implant in a dense fibrous capsule that isolates the device from surrounding host tissue. Macrophages persist for the lifetime of the implant. Medical device failure is often caused by fibrous encapsulation.

The mechanisms that lead to fibrous encapsulation are not well-understood. Cellular FLICE-like inhibitory protein (cFLIP) plays a role in inhibiting apoptosis in cancer cell models. Inhibiting cFLIP in macrophages reduced fibrosis in lung injury models.

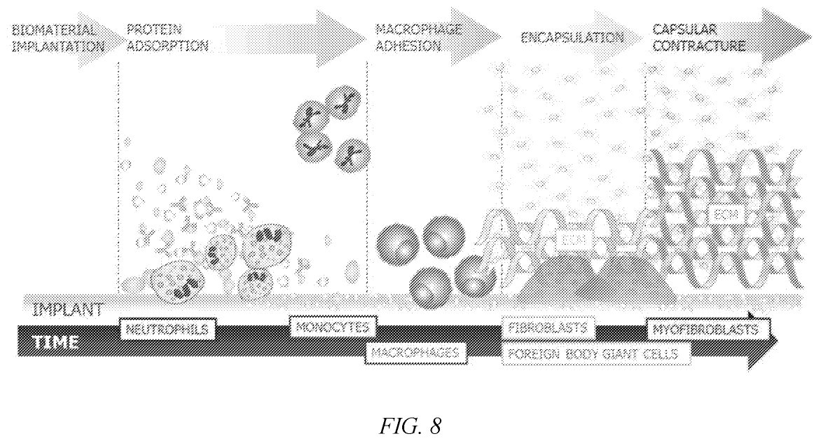

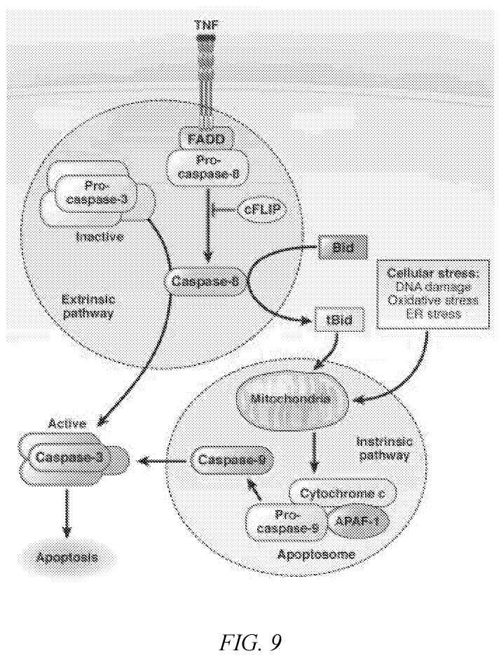

Caspase-mediated extrinsic pathway: CD95, or FasL, initiates the extrinsic apoptotic pathway by binding the Fas receptors on the cell surface. (FIGS. 8 and 9) Once bound, the Fas-associated death domain (FADD) is recruited to the site intracellularly and initiates the binding of procaspase-8 to the complex. The intact death-inducing signaling complex (DISC) induces caspase-8 activity thus activating the effector caspase-3 leading to apoptosis of the cell.6 cFLIP inhibits the activation of caspase-8, which then allows the cell to evade death even when the complex death complex is initiated.

It is shown herein that (1) cFLIP is upregulated in macrophages causing their persistence in the FBR which leads to fibrous encapsulation and (2) inhibiting cFLIP in macrophages abrogates fibrosis in the FBR.

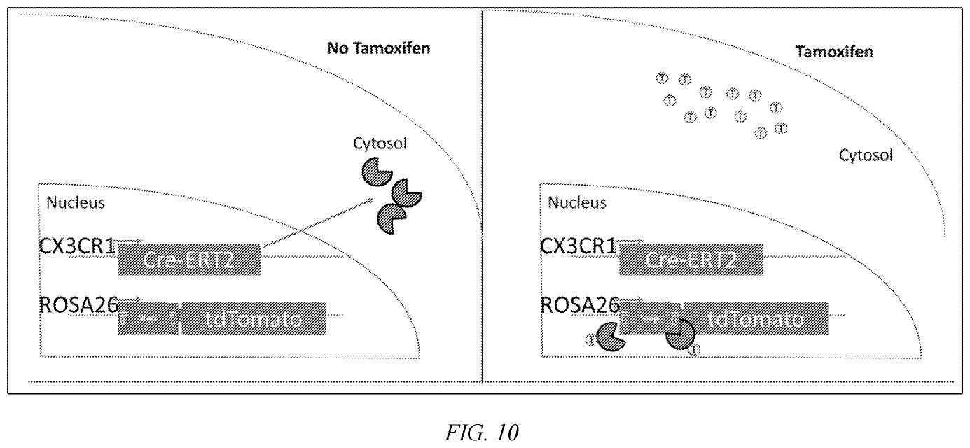

A transgenic murine model was developed for CRE-DRIVER. (See FIG. 10). Cx3cr1 containing immune cells (which includes macrophages and monocytes) inserted with inducible CreER-gene. Transgenic mice utilized to test 1) deletion of the gene Cflar that encodes for cFLIP and 2) lineage tracing of macrophages in cells containing active Cx3cr1 locus. A first group of mice were the Cx3cr1^{CreERT2}×tdTomato Reporter Mice. These mice showed tamoxifen induced fluorescence. Macrophages and monocytes and respective daughter cells fluoresce with tdTomato and lineage tracing. A second group of mice were the Cx3cr1^{CreERT2}×cFLIP Mice (cFLIP^{Δ/Δ}). These mice showed tamoxifen-induced gene reduction. Macrophages and monocytes and respective daughter cells no longer carry Cflar gene that encodes for cFLIP. There was temporal control on reduction.

FIG. 10 provides a schematic of Cx3Cr1 Cre-driven transgenic murine model that is initiated by tamoxifen treatment. When mice are injected with tamoxifen, the Cre-enzymes that are naturally stored in the cytosol are able to bind to the chemical and pass through the nuclear membrane. The enzymes then cleave away the floxed genes, in this instance removing the stop codon and allowing tdTomato expression in cells.

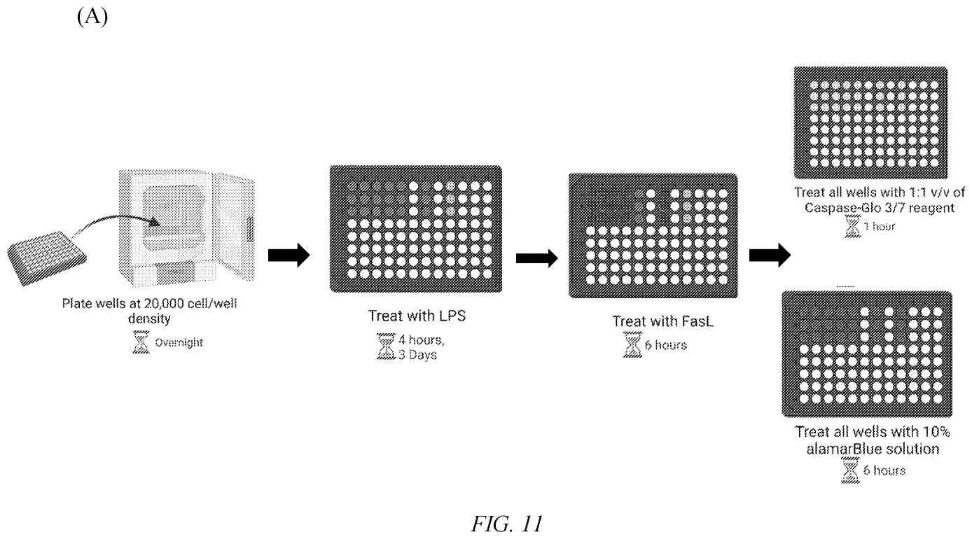

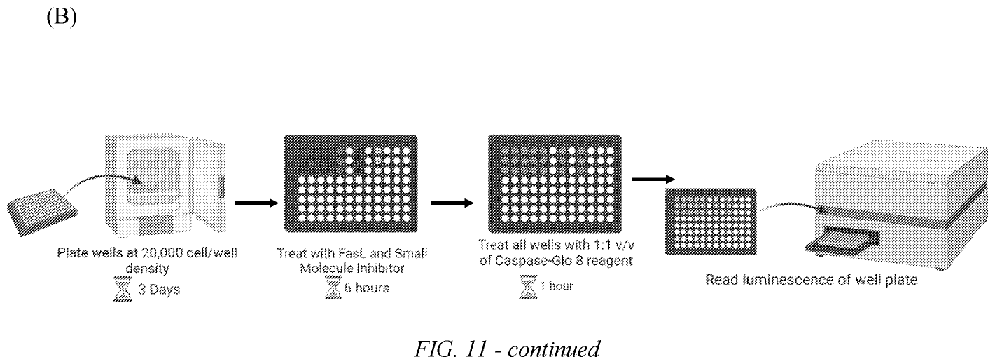

In vitro assessments of cell behavior was conducted as outlined in FIG. 11. FIG. 11A shows an in vitro experimental protocol. J774A.1 macrophage cell line was incubated overnight at 20,000 cells/well and treated with lipopolysaccharide (LPS) for 4 hours or 3 days. Exogenous FasL was then added to initiate the extrinsic apoptotic pathway for 6 hours before caspase activity (Caspase-Glo 3/7) and metabolic activity (alamarBlue) assays were performed to study the differences in cell behavior after exposure to each stimulant.

FIG. 11B shows an in vitro experimental protocol for small molecule testing. J774A.1 macrophage cell line was incubated overnight at 20,000 cells/well and cultured in complete media for 3 days. Exogenous FasL was then added to initiate the extrinsic apoptotic pathway with or without a small molecule inhibitor for 6 hours before a caspase activity (Caspase-Glo 8) assay was performed to study the differences in cell behavior after exposure.

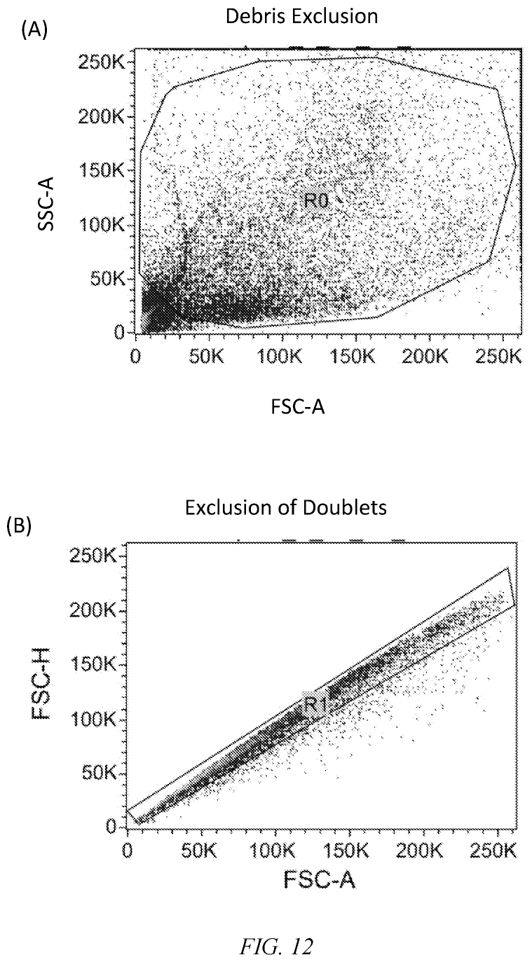

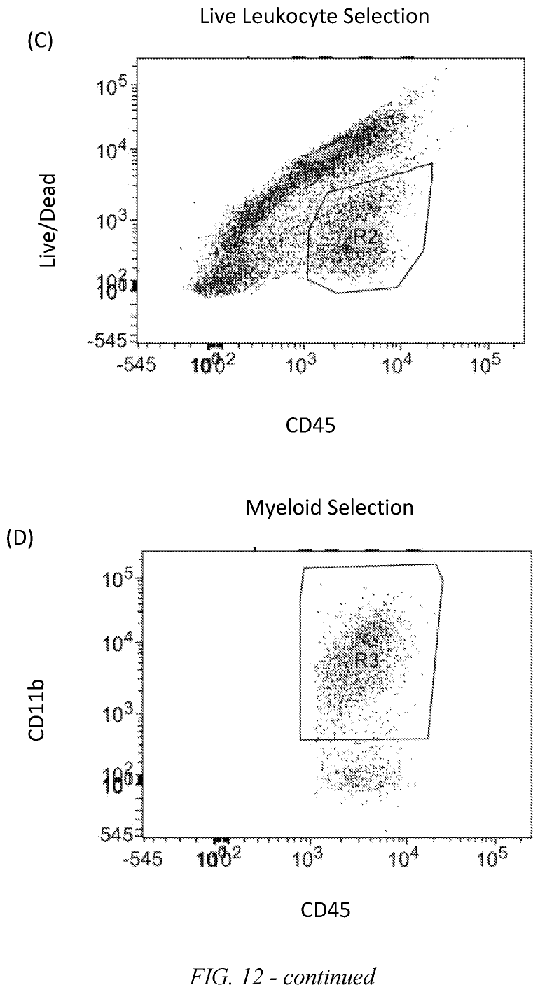

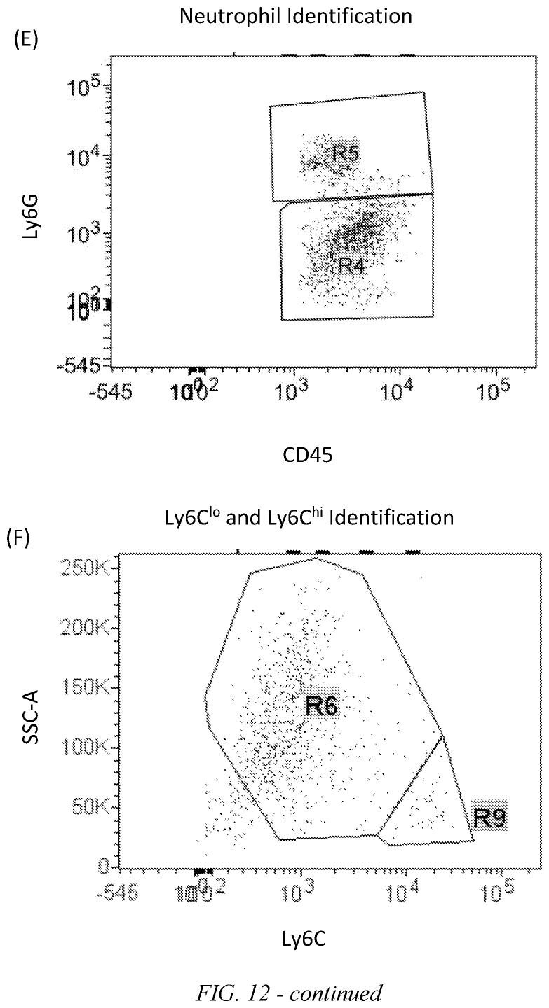

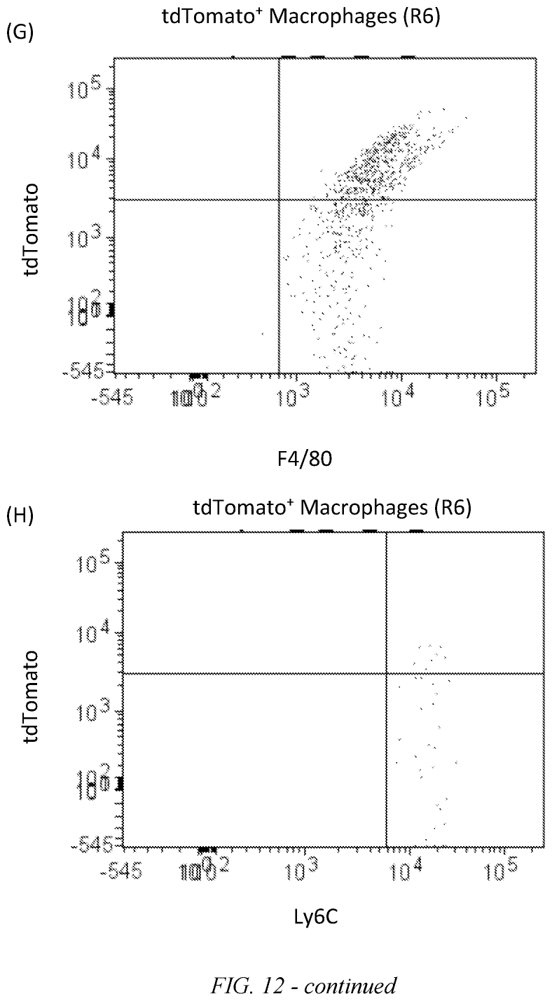

Cell identification can be achieved through fluorescent staining as shown in FIG. 12. A gating strategy was utilized for distinguishing immune cell types from the surrounding implant tissue area. Flow cytometry was used to distinguish immune cell types: CD45+ leukocytes, CD11b+ myeloid cells, Ly6G+ neutrophils, Ly6C+ monocytes, F4/80+macrophages, and Zombie Dye to distinguish dead cells. tdTomato positivity was achieved via tamoxifen injections given to the reporter transgenic mouse model.

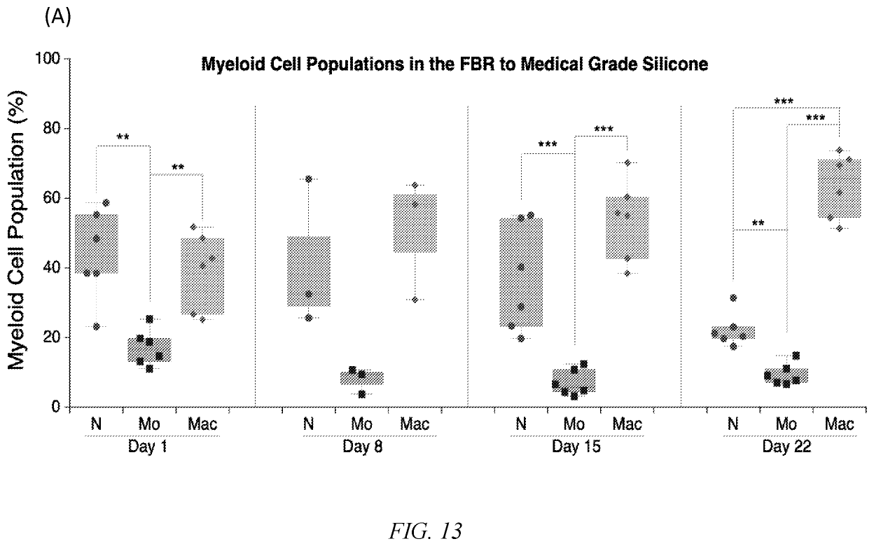

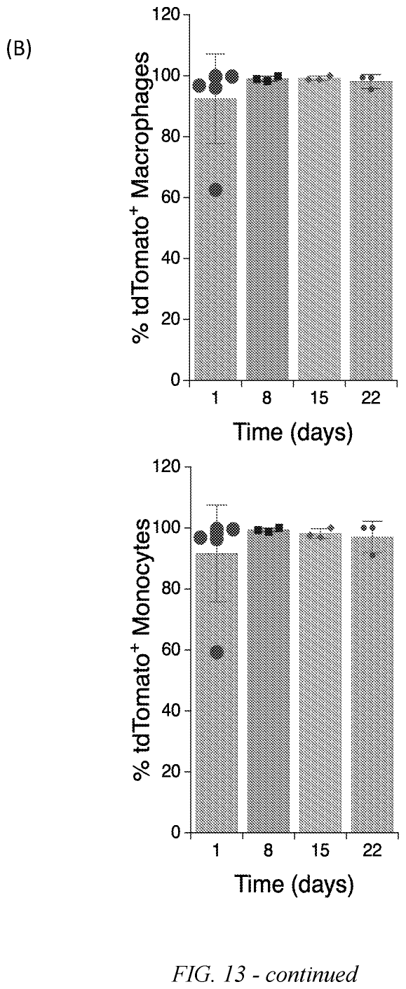

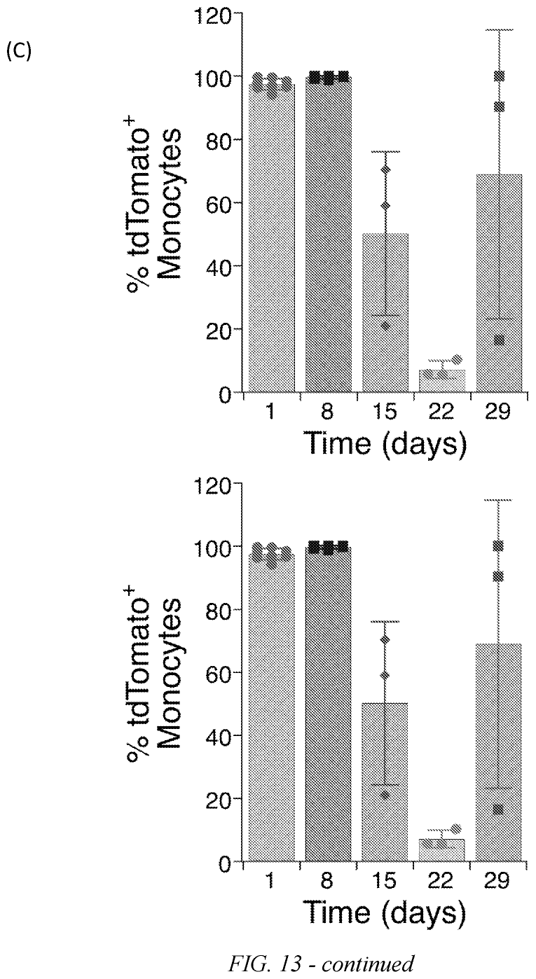

Myeloid cell populations and tdTomato+ macrophages and monocytes in the FBR were investigated. (See FIG. 13) Macrophage frequency is shown to be higher relative to other myeloid cells, and nearly all monocytes and macrophages are tdTomato positive with tamoxifen (TMX) treatment. FIG. 13(A) shows the temporal frequency of neutrophils (N), monocytes (Mo), and macrophages (Mac). FIG. 13(B) shows that nearly all macrophages and monocytes are tdTomato⁺ with continual TMX treatment. FIG. 13(C) demonstrates that tdTomato⁺ macrophages and monocytes decrease over time, but by day 29 over half of the macrophages and monocytes are persistent; TMX treatment up to day 7. *p<0.05; **p<0.01; ***p<0.001; symbol above a column compares to day 1.

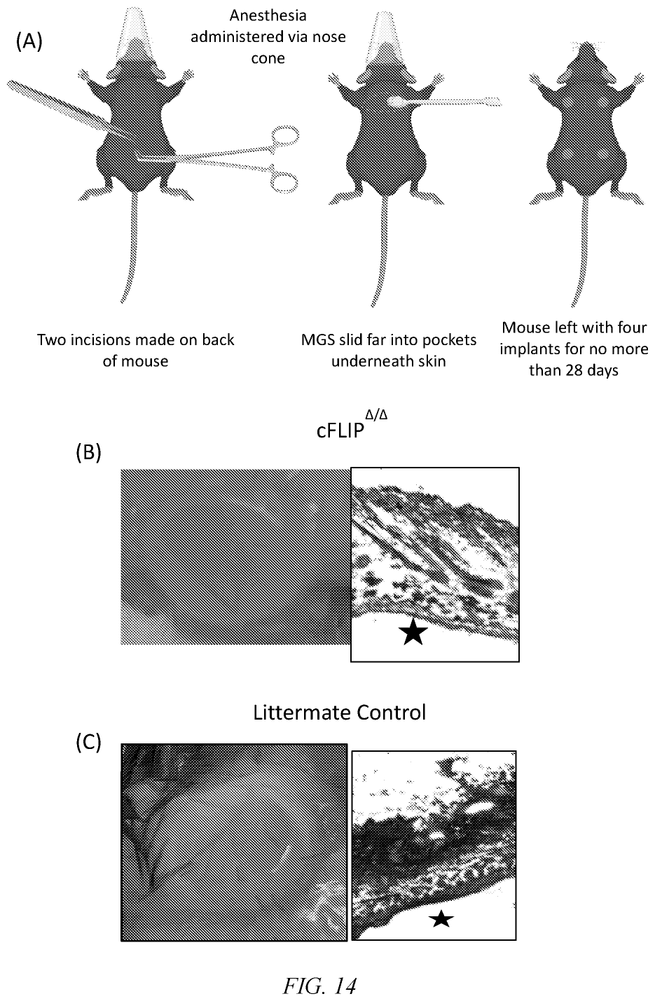

Subcutaneous implants of silicone were introduced into a mouse model. A cFLIP conditional knockout model qualitatively indicates a looser fibrous capsule at 28 days post-implantation with tamoxifen treatment. FIG. 14(A) shows a schematic of implant surgeries on mice. FIG. 14(B) shows implant and histology samples from a cFLIP knockout model. FIG. 14(C) shows implant and histology sample from a littermate control model. A fibrous capsule can be seen immediately above the star at the implant site.

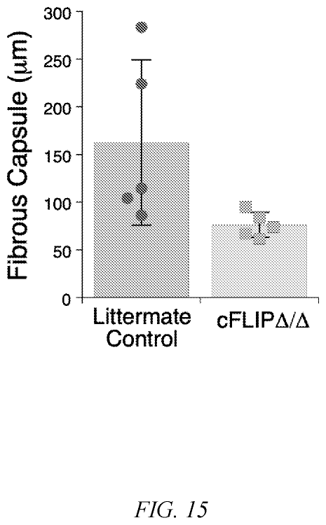

Capsule thickness was investigated. Specifically, a quantification of capsule thickness=on day 29 post-implantation was studied. (See FIG. 15) The capsule thickness was lower (p=0.08) different in cFLIP^{Δ/Δ} mice, which is consistent with the histology images, compared to the littermate control.

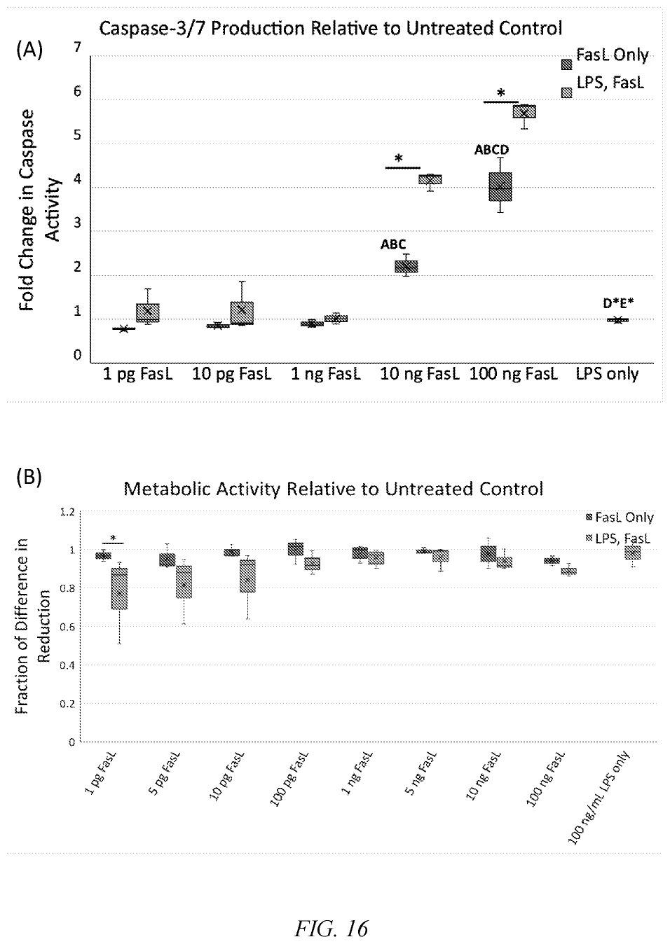

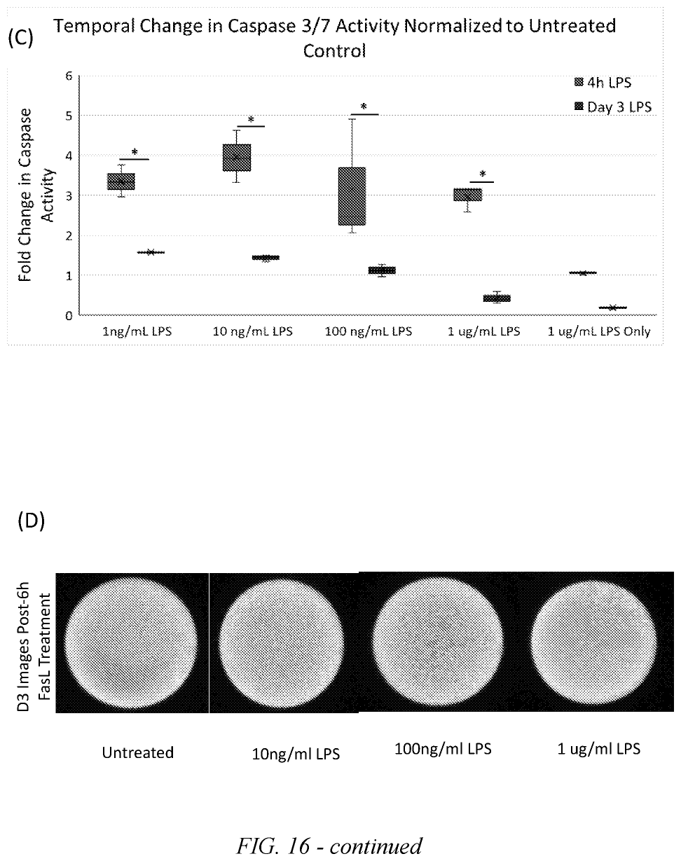

An in vitro model was used to recapitulate cell persistence. The model used a J774A. 1 cell line sensitive to stimuli causing pro-survival behavior. Caspase production was significantly decreased with long-term exposure to an inflammatory stimulant which will allow for testing small molecules to reverse the induced pro-survival behavior in future experimentation. FIG. 16(A) compares the fold-change in caspase 3/7 activity between each condition relative to untreated control conditions; ABCDE signifies p<0.05 between indicated FasL concentrations; D*E* indicates significant differences from both FasL Only and LPS, FasL conditions. FIG. 16(B) indicates metabolic activity using an alamarBlue assay of each tested condition relative to untreated control conditions. FIG. 16(C) shows differences in caspase 3/7 activity when cells were stimulated with LPS for either 4 hours or 3 days then subjected to FasL stimulation at a concentration of 100 ng/ml for 6 hours. FIG. 16(D) shows qualitative images of each well plate after 3 days in varied concentrations of LPS. N=3; *p<0.05.

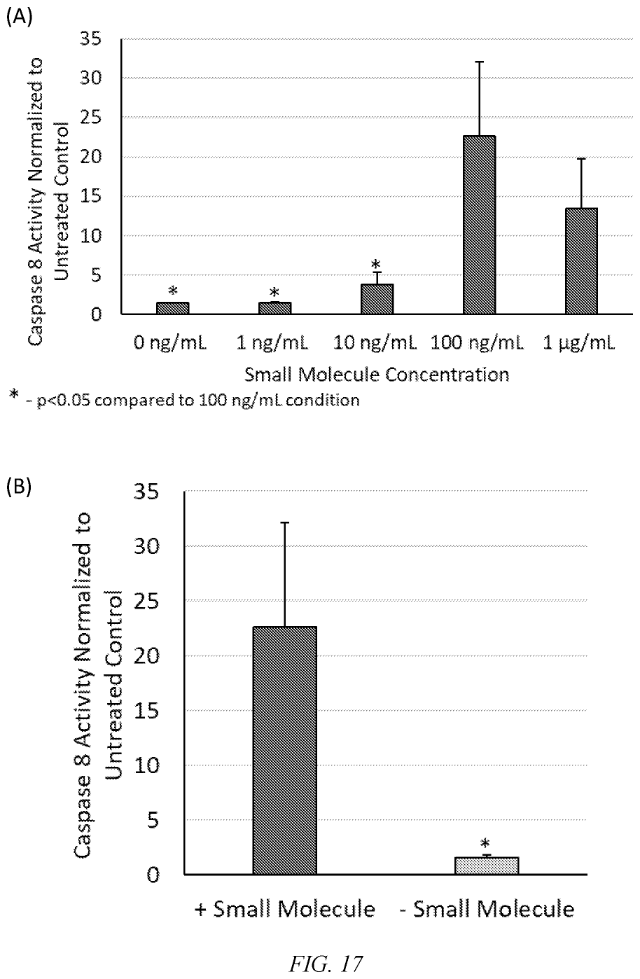

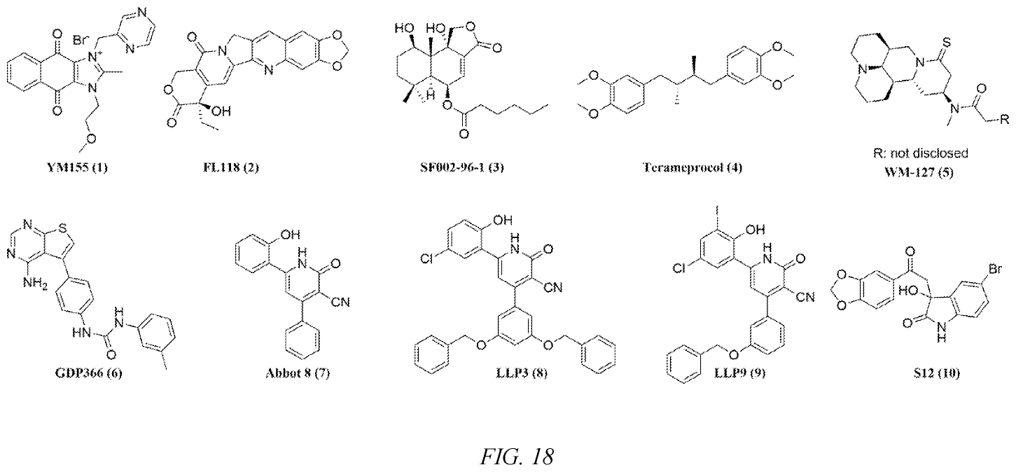

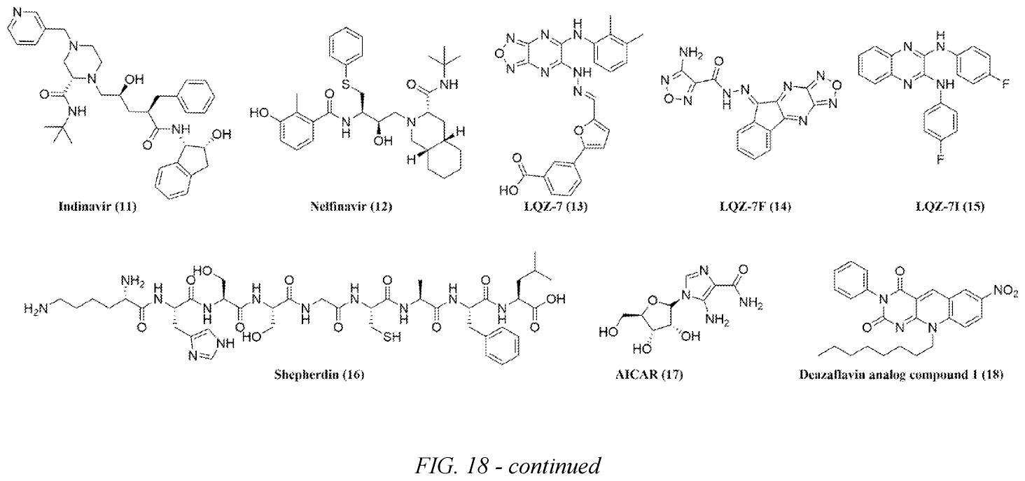

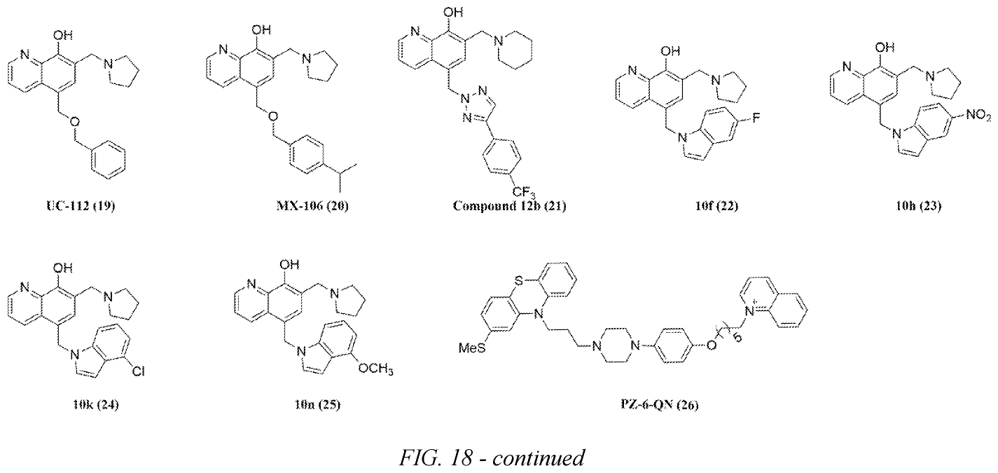

Lastly, tests were performed utilizing a small molecule inhibitor (YM155) of CFLIP. Small molecule exposure was demonstrated to lead to increased caspase 8 activity. FIG. 17(A) Cells treated with 100 ng/ml of small molecule expressed a fold change of about 23 times greater than the untreated control. FIG. 17(B) Confirmation that the small molecule is causing increased caspase activity rather than the solvent it is dissolved in. N=3.

Nearly all macrophages and monocytes are tdTomato⁺ confirming they are Cx3cr1⁺. Weekly Tmx treatment is sufficient to continually label nearly all macrophages and monocytes. Persistent macrophages and monocytes are observed at day 29 and make-up over half of the cells. Using conditional cFLIP knockout murine model (cFLIP^{Δ/Δ}), conditionally knocking down cFLIP in Cx3cr1⁺ cells does not affect capsule thickness, but leads to a less dense capsule. An in vitro model with LPS stimulation decreased cell sensitivity towards pro-apoptotic stimuli allowing further investigation into small molecules to reverse persistent behavior. The in vitro model with small molecule treatment significantly increased cell sensitivity towards pro-apoptotic stimuli shown with increased caspase activity.

### Definitions

The term “administration” and variants thereof (e.g., “administering” a compound) in reference to a compound of the invention means introducing the compound into the system of the subject in need of treatment.

As used herein, the term “composition” is intended to encompass a product comprising the specified ingredients in the specified amounts, as well as any product which results, directly or indirectly, from combination of the specified ingredients in the specified amounts.

As used herein, “treatment” refers to obtaining beneficial or desired clinical results. Beneficial or desired clinical results include, but are not limited to, any one or more of: alleviation of one or more symptoms (such as FBR), diminishment of extent of the formation of a fibrous capsule, stabilized (i.e., not worsening) state the formation of a fibrous capsule, preventing or delaying spread the formation of a fibrous capsule, preventing or delaying occurrence or recurrence or slowing of the formation of a fibrous capsule progression, amelioration of the state. The methods of the invention contemplate any one or more of these aspects of treatment.

A “pharmaceutically acceptable” component is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

A medical device is an instrument, tool, machine, test kit, or implant that is used to prevent, diagnose, or treat disease or other conditions. A medical device is defined as implantable if it is either partly or totally introduced, surgically or medically, into the human body and is intended to remain there after the procedure. As defined by the FDA, an implantable medical device is a device that is placed into the human body for a period of 30 days or more.

According to the National Cancer Institute, a small molecule is a drug that can enter cells easily because it has a low molecular weight. Once inside the cells, it can affect other molecules, such as proteins, and may cause cancer cells to die. This is different from drugs that have a large molecular weight, which keeps them from getting inside cells easily. Many targeted therapies are small-molecule drugs.

Encapsulation is the effective surrounding of a therapeutic agent that provides protection and/or release of the therapeutic agent. Encapsulation is an effective tool for targeted deliveries of drugs and sensitive compounds. The agent used for encapsulation is the encapsulating agent. Encapsulation approaches are based on barriers made from (bio) polymers, liposomes, multiple emulsions, etc.

Sustained release technology is a class of technology characterized by slowly-releasing specific active substances into a target medium to keep a certain concentration in the system within valid time. As used herein, the term includes extended-release and controlled release of a drug or dosage.

Biodegradable polymeric materials have been used for extended release. One such example is polylactic acid copolymer, which degrades to lactic acid and eliminates the problem of retrieval after implantation. Other polymers for drug formulations include polyacrylate, methacrylate, polyester, ethylene-vinyl acetate copolymer (EVA), polyglycolide, polylactide, and silicone. Of these, the hydrophilic polymers, such as polylactic acid and polyglycolic acid, erode in water and release the drug gradually over time. A hydrophobic polymer such as EVA releases the drug over a longer duration time of weeks or months. The rate of release may be controlled by blending two polymers and increasing the proportion of the more hydrophilic polymer, thus increasing the rate of drug release. [Chapter 17. Modified-Release Drug Products. In: Shargel L, Wu-Pong S, Yu AC. eds. Applied Biopharmaceutics & Pharmacokinetics, 6e. McGraw Hill; 2012.]

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

TABLE 1
Survivin Inhibitors

| Inhibitor | Target Protein/Pathway |
|---|---|
| YM155 | Survivin at mRNA, protein, and |
| | transcription levels; cFLIP |
| FL118 | Survivin at mRNA and protein levels |
| SF002-96-1 | Survivin by inhibition of STAT3 and NF-KB |
| Terameprocol | Survivin |
| WM-127 | Survivin |
| GDP366 | Survivin at gene and protein levels |
| Abbot 8 | Survivin dimerization |
| LLP3 | Survivin dimerization |
| LLP9 | Survivin dimerization |
| S12 | Survivin dimerization |
| Indinavir | Survivin protein |
| Nelfinavir | Survivin protein |
| LQZ-7 | Survivin dissociation and degradation |
| LQZ-7F | Survivin dissociation and degradation |
| LQZ-7I | Survivin dissociation and degradation |
| Shepherdin | Survivin interactions with Hsp90 |
| AICAR | Sruvivin interactions with Hsp90 |
| Deazaflavin analog | Survivin interaction with Smac |
| compound 1 | |
| UC-112 | Survivin degradation through |
| | ubiquitin-mediated pathway |
| MX-106 | Survivin degradation through |
| | ubiquitin-mediated pathway |
| Compound 12b, | Survivin degradation through |
| 10f, 10h, 10k, 10n | ubiquitin-mediated pathway |
| PZ-6-QN | Survivin interaction with Smac |
| | Adapted from: Albadari N, Li W. Survivin |
| | Small Molecules Inhibitors: Recent Advances |
| | and Challenges. Molecules. |
| | 2023 Feb. 1; 28(3): 1376. |
| | doi: 10.3390/molecules28031376. |
| | PMID: 36771042; PMCID: PMC9919791. |

1. A coating for an implantable medical device comprising YM155 and an encapsulating agent or hydrogel, wherein the encapsulating agent or hydrogel is capable of immobilization on the surface of an implantable medical device.

2. The coating according to claim 1 wherein the YM155 is conjugated to the encapsulating agent.

3. The coating according to claim 1 wherein the encapsulating agent is a biodegradable polymer.

4. The coating according to claim 1 wherein the encapsulating agent is an acrylate.

5. A method of preparing a medical device for implantation comprising the step of coating the medical device with a coating according to claim 1.

6. A coating for an implantable medical device comprising a small molecule inhibitor of survivin selected from the group consisting of YM155, FL118, SF002-96-1, Terameprocol, WM-127, GDP366, Abbot 8, LLP3, LLP9, S12, Indinavir, Nelfinavir, LQZ-7, LQZ-7F, LQZ-7I, Shepherdin, AICAR, Deazaflavin analog compound 1, UC-112, MX-106, Compound 12b, Compound 10f, Compound 10h, Compound 10k, Compound 10n, PZ-6-QN and combinations thereof and an encapsulating agent or hydrogel, wherein the encapsulating agent or hydrogel is capable of immobilization on the surface of an implantable medical device.

7. The coating according to claim 6 wherein the small molecule inhibitor of survivin is conjugated to the encapsulating agent.

8. The coating according to claim 6 wherein the encapsulating agent is an acrylate.

9. The coating according to claim 6 wherein the encapsulating agent is a biodegradable polymer.

10. A method of preparing a medical device for implantation comprising the step of coating the medical device with a coating according to claim 6.

11. (canceled)

12. (canceled)

13. (canceled)

14. (canceled)

15. A method of inhibiting or reducing the FBR responsive to the implantation of a medical device by delivering to the site of implantation a small molecule inhibitor of survivin selected from the group consisting of YM155, FL118, SF002-96-1, Terameprocol, WM-127, GDP366, Abbot 8, LLP3, LLP9, S12, Indinavir, Nelfinavir, LQZ-7, LQZ-7F, LQZ-7I, Shepherdin, AICAR, Deazaflavin analog compound 1, UC-112, MX-106, Compound 12b, Compound 10f, Compound 10h, Compound 10k, Compound 10n, PZ-6-QN and combinations thereof.

16. The method according to claim 15 wherein the small molecule inhibitor is encapsulated in a sustained-release encapsulating agent.

17. (canceled)

18. (canceled)

19. (canceled)
